(19) United States
(12) Patent Application Publication
(10) Pub. No.: US 2006/0257958 A1
(43) Pub. Date: Nov. 16, 2006

(54) Magnetically-assisted test strip cartridge and method for using same

(75) Inventor: John G. Bruno, San Antonio, TX (US)

Correspondence Address: LOEFFLER JONAS & TUGGEY, LLP, 755 EAST MULBERRY STREET, SUITE 200, SAN ANTONIO, TX, 78212, US

(73) Assignee: PRONUCLEOTEIN BIOTECHNOLOGIES, LLC

(21) Appl. No.: 11/433,284

(22) Filed: May 12, 2006

Related U.S. Application Data
(60) Provisional application No. 60/680,979, filed on May 13, 2005.

(51) Int. Cl. G01N 33/00; G01N 33/53; C12M 1/34

(52) U.S. Cl. 435/7.93; 435/287.2; 977/900

(57) ABSTRACT

The present invention provides a small (business card-sized) disposable mesofluidic or microfluidic plastic cartridge containing several straight microchannels potentially filled with culture media, solubilizing reagents (e.g., detergents) nitrocellulose paper strip, gel or other matrix materials and lyophilized paramagnetic microbeads or microparticles coated with antibodies, nucleic acid aptamers, oligonucleotides, or other types of proteins or other receptors for capture, concentration and ultrasenstive detection of target analytes in environmental, food, animal, or clinical body fluid samples.

This application is based upon and claims priority from U.S. Provisional application Ser. No. 60/680,979, which is incorporated herein by reference.

### BACKGROUND OF THE INVENTION

1. Field of the Invention

The present invention relates to the fields of food safety assessment, environmental field detection, veterinary, agricultural, and point-of-care clinical sensors and diagnostics. These areas require ultrasensitive and rapid assays for analytes such as bacterial and viral pathogens or parasites and clinically important analytes or biomarkers in order to detect the few microbes or molecules capable of causing disease or indicating the early presence of disease or potential toxicity and harm. Yet, these areas require analysis of small sample quantities (e.g., 100 μL or lesser quantities) in a rapid fashion (within minutes) on-site or at the bedside to avoid costly and time-consuming transportation to central laboratories. These areas are also plagued with “dirty” samples and complex matrices such as food samples, blood, urine, and body fluids or turbid water samples.

2. Background Information

Considering the difficult requirements for environmental and clinical detection and diagnostics, the present invention strives to integrate the highest affinity receptors (aptamers and antibodies) with the most sensitive fluorescence technology available (e.g., quantum dots “QD's” or fluorophore-filled plastic nanoparticles) and a simple magnetic concentration and separation approach to amplify and purify the sample in a small (credit card-sized) format that is disposable and could be hermetically sealed for chain of custody requirements, if desired. For a number of applications, assay speed and ultrasensitivity are desired. In the food safety arena, extreme speed and sensitivity are desirable to minimize or eliminate the enrichment culture phase of detection, because during that period of hours to days when bacteria are being cultured from a food sample, potentially contaminated foods can be sold and consumed by humans, possibly resulting in illness or death for the consumer. In biowarfare or bioterrorism detection, speed and sensitivity are desired to enable a soldier or first responder to don protective masks, gloves, overgarments or other gear and thereby minimize or prevent exposure to deadly pathogens, toxins or other substances. Although, the present invention, magnetically-assisted test strip (“MATS”) is intended to combine aptamer-MB or antibody-MB and QD-based assays into a simple cartridge format, other visible or less sensitive assays (e.g., competitive displacement FRET) assays are also possible within the framework of a MATS cartridge and are therefore claimed.

Several related inventions such as a line of electrochemiluminescence (“ECL”) sensors rely on micron-sized magnetic beads (“MB's”) to aid in purifying and concentrating target analytes from the sample matrix. ECL devices are generally large (greater than a cubic foot). Only recently, have ECL devices been developed that are handheld or compact and portable. Others have developed larger immunomagnetic (antibody-MB) separation and detection devices that are of the table-top variety consisting of a cubic foot or more in volume. Such devices were designed and constructed to handle large sample volumes (e.g., ≧10 mL).

Existing technologies have a similar concept, but with several important differences: 1) do not mention QDs as possible fluorophores in their system, 2) assay pathways are complex and tortuous with numerous bifurcations, whereas the MATS microchannels are typically straight or uncomplicated, 3) existing devices employ microscopic valves, which are not necessary in the linear fluid channel design of the MATS especially when a nitrocellulose paper strip or other matrix material is added in the channels.

Other existing art utilizes MBs in biosensor assay cartridges in various ways. Their detection schemes do not involve fluorescence, but are based on other visual means of detection or measurement of the magnetic field around the MB before and after binding of an analyte, which has certain advantages in terms of simplicity, but may not be as sensitive as the fluorescence intensity or ECL methods and is certainly not as sensitive as the time-resolved fluorescence methods.

### SUMMARY OF THE INVENTION

The present invention is referred to as a “Magnetically Assisted Test Strip” (“MATS”), which is reminiscent of immunochromatographic test strips employing latex microbeads, except that by using MBs, the operator has control over the flow rate of the particles along their path toward the formation of a sandwich, FRET, or other type of assay on the MB's surface. Control of the MB flow rate is imposed via an external magnetic field such as a permanent magnet or electromagnet that moves the MBs along the channel and halts the MBs wherever critical reagents or sample components must be picked up. The MATS would typically be encased in a plastic cartridge and is therefore referred to as the “MATS cartridge.”

The present invention provides a small (business card-sized) disposable mesofluidic or microfluidic plastic cartridge containing several straight microchannels potentially filled with culture media, solubilizing reagents (e.g., detergents) nitrocellulose paper strip, gel or other matrix materials and lyophilized paramagnetic microbeads or microparticles coated with antibodies, nucleic acid aptamers, oligonucleotides, or other types of proteins or other receptors for capture and concentration of target analytes in environmental, food, animal, or clinical body fluid samples. Target analytes in fluids are allowed to wet and interact with the lyophilized capture reagent-magnetic beads and are then moved to a second position by means of an external magnetic field. Along the way, any debris or interfering material that was in the sample tends to be left behind, thereby purifying and concentrating the target. At the second position, the captured target analytes on the surface of the magnetic beads interact with secondary or “reporter” reagents, such as fluorescent dye-, fluorophore-, fluorescent protein-, quantum dot (“QD”)-, nanoparticle-(“NP”), colloidal gold-, or enzyme-conjugated antibodies, nucleic acid aptamers or fluorescence resonance energy transfer (“FRET”) reagents such as FRET-aptamers or molecular beacons. In effect, a mobile or “rolling” sandwich assay is formed on the magnetic bead's surface. These reactants on the magnetic bead are further translated by the external magnetic field to a third position, which is a transparent miniature detection window for viewing of the resulting fluorescence or other visible reactions. Again, in the process of moving from the second to the third position, unwanted materials such as unbound reporter reagents tend to be left behind. In another configuration, the magnetically immobilized beads in the detection window could be back-flushed and washed, if desired, by means of a buffer-loaded syringe or small pump. Finally, results are detected or quantified by fluorometer, fluorescence intensity, spectrofluorometry features, fluorescence lifetime analysis, spectrophotometry, spectrofluorometer, time-resolved fluorometer, photodiode, photodiode array, charge-couple device (“CCD”) camera, complementary metal oxide semiconductor (“CMOS”), photomultiplier tube (“PMT”), or visual assessment of the magnetic bead-target complexes in the miniature detection window. Because, several types (sizes) of quantum dots can be simultaneously detected based on their fluorescence emission, simultaneous multiplexed detection of several target analytes is also possible. Finally, a means of covalently attaching QDs or other fluorophores to the 3′ end of DNA aptamers is disclosed for conjugate development and use in the detection assays and cartridges.

The void or working volume of the MATS cartridge is small and typically in the range of 50 μL to 100 μL. With such a reduced sample volume, it is necessary to concentrate all the available analyte to a small point, such as the surface of a MB, and to use an ultrasensitive detection technology, such as QD technology. Approximately 60 zeptomolar (60×10⁻²¹ M) levels have been detected from time-resolved immuno-QD assays for prostate specific antigen (“PSA”). Even simple fluorescence intensity readings employing QDs have proven quite sensitive. There is the potential for detecting as low as one bacterium with antibody-QD conjugates in some systems.

Moreover, there has been discrimination of fluorescence signals from four different biotoxin assays using various types of QDs in the same microtitre well, thus supporting the claim that multiplex analyses are possible in the same microchannel of a MATS cartridge. Hence, if a MATS cartridge is designed with five different microchannels leading to five different detection windows and four different analytes can be detected in each by multiplexing and detecting four different emission wavelengths at the same time, then a panel of twenty different tests can be run in a single MATS cartridge within minutes.

If the target analyte is entangled or encased within a tissue matrix or cell, such as Leishmania bacteria in a macrophage, a malaria parasite within an erythrocyte or an intracellular protein of interest, a MATS cartridge can be designed with a pre-processing “chamber” in its flow path for extricating the target or dissolving away the matrix to reveal the target via dried chaotropic agents, detergents, or enzymes. Likewise, bacterial numbers can be increased to improve odds of detection in an enrichment culturing pre-processing chamber of a MATS cartridge. Such an enrichment culturing chamber or region in the MATS flow path would contain lyophilized nutrients that would support the growth and replication of bacteria upon wetting by the sample. Incorporation of an enrichment culturing chamber or region and dried culture media such as nutrient broth (“NB”), tryptic soy broth (“TSB”), brain heart infusion (“BHI”) broth, other common, or specialized microbiological culture media prior to the start of the rolling sandwich assays in the channels of the MATS cartridge can serve to increase numbers of bacteria by supporting bacterial replication after a sufficient enrichment period from foods, body fluids, environmental or other samples prior to analysis.

A solubilizing or dissolution chamber or area may be incorporated into the channels. A solubilizing or dissolution chamber or area would contain ionic or nonionic detergents, enzymes, or chaotropic agents such as sodium dodecyl sulfate (“SDS”), Triton detergents (e.g., Triton X-100), Tween detergents (Tween 20, Tween 80, etc.), trypsin, proteinase K, magnesium chloride (MgCl₂) or other solubilizing and degrading agents in sufficient quantities to release bacteria, parasites, or viruses from meats, tissue particles, or cells, etc. or to release detectable levels of target proteins or other antigens prior to the rolling sandwich assays in the channels of the MATS cartridge.

MATS cartridges may employ a number of QD-conjugated or other fluorophore-conjugated DNA aptamers, such as using covalent attachment chemistry for proteins to the N⁶ primary aryl amine of the terminal 3′ adenine added by Taq polymerase during the polymerase chain reaction (“PCR”) via bifunctional aldehydes (e.g., glutaraldehyde), succinimides, or other bifunctional linkers. Alternatively, adenine, cytosine, and guanine added by terminal deoxynucleotide transferase (“TdT”) to blunt-ended double-stranded (“ds”) DNA can be used as well, since the primary amines in these terminal nucleotides are the only susceptible groups for chemical attachment in otherwise ds DNA. Attachment to the 3′ end of aptamers and other types of DNA probes is desirable because it confers greater serum stability. Use of 3′-adenine attachment chemistry arises by involving N⁶ of adenine and bifunctional aldehydes (e.g., glutaraldehyde) or other amine-reactive homobifunctional linkers or heterobifunctional linkers (such as succinimide linkers) for any DNA aptamer-QD or other oligonucleotide-QD, fluorophore, or nanoparticle linkages. A 3′-adenine overhang can be obtained by one round of polymerase chain reaction (PCR) using conventional Taq DNA polymerase. Hence a complementary strand to the intended aptamer or oligonucleotide sequence is used followed by one round of PCR, resulting in a 3′-adenine overhang. N⁶ of the adenine is therefore the only susceptible target on the otherwise double-stranded DNA where bifunctional aldehydes or other linkers can attack for conjugation of QDs, nanoparticles, fluorescent proteins, or other fluorophores. The resulting DNA-fluorescent conjugate entity can be made single-stranded by that application of heat or denaturing agents such as urea and purified by size-exclusion chromatography, gel electrophoresis or other standard molecular separation techniques and used in assays within a MATS cartridge.

This same chemical attachment strategy for proteins to ds DNA PCR products can be applied to the chemical conjugation of ds aptamers to amine-QD, carboxyl-QD or other derivatized QD conjugates. The N⁶ aryl amine of adenine may be used for attachment to amine-QDs by means of a carbodiimide binfunctional linker. The aptamer-QDs made by this method can be valuable reagents in the sandwich and other assays employed in a MATS cartridge. Therefore, the chemical conjugation method using primary amines from adenine, cytosine, or guanine with aldehyde or succinimide-based bifunctional linkers and derivatized QDs is claimed as part of the MATS assay methodology.

The method herein may also use TdT to add adenine, cytosine, or guanine residues to blunt-ended double stranded DNA aptamers or oligonucleotides followed by attachment of bisaldehydes (e.g., glutaraldehyde) or other suitable amine-reactive homobifunctional or heterobifunctional linkers for conjugation of the aptamer or oligonucleotide at its 3′ end to a QD, fluorophore, fluorophore-filled nanoparticle, or fluorescent protein at the vulnerable primary aryl amine of adenine, cytosine, or guanine. The resulting DNA-fluorescent conjugate entity can be made single-stranded by the application of heat or denaturing agents such as urea and purified by size-exclusion chromatography, gel electrophoresis or other standard molecular separation techniques.

### BRIEF DESCRIPTION OF THE DRAWINGS

FIG. 1. is an exploded perspective view of the components and design of a MATS cartridge.

FIG. 2. schematically illustrates the use of the MATS cartridge.

FIG. 3. is a schematic illustration of dissolving and enrichment culture pre-treatment chambers that may be present in an alternative embodiment of the MATS cartridge.

FIG. 4. is a schematic illustration of the chemistry for conjugation of DNA aptamers.

### DETAILED DESCRIPTION OF THE PREFERRED EMBODIMENT

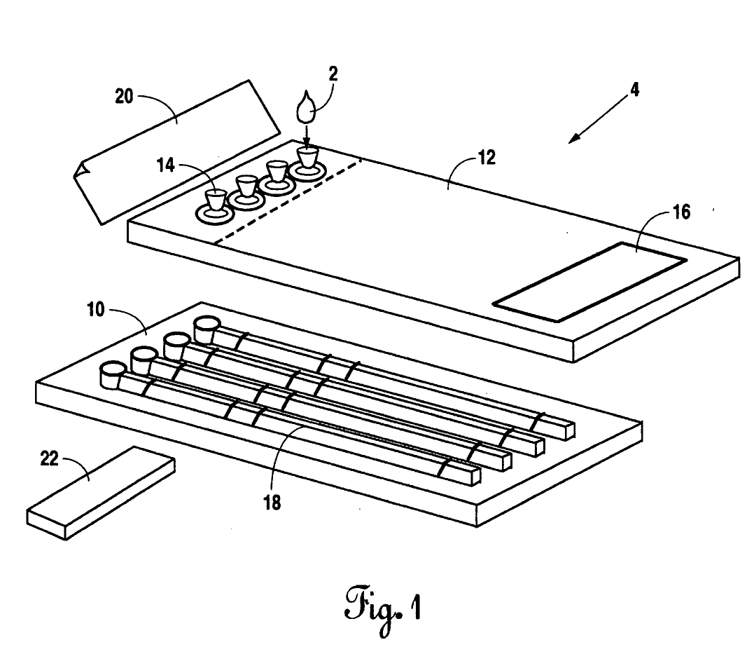

Referring to the figures, FIG. 1. is an exploded perspective view of the components and design of a MATS cartridge (4). The cartridge (4) body is fabricated in two halves (10 and 12) typically composed of plastic, silica, metals, or other substrates for the purpose of magnetic transport of receptor (antibody, aptamer, or other receptor) conjugated-magnetic microbeads (not shown). The two halves (10 and 12) can be joined by way of heat (such as fusion, laser, welding, ultrasound, microwaves, or other means of melting the halves together), adhesives (such as glue or tape), mechanical fasteners (such as bolts, screws, rivets, clamps, or weight), or other means (such as hook and loop fasteners or friction). The top half (12) contains sample ports (14) and may be opaque except for the detection window(s) (16). The sample port (14) may use gravity-assisted flow, capillary action, slight suction or slight pressure to help introduce a liquid sample (2) to the channels (14). Gaskets can be added between the top and bottom halves (10 and 12) to prevent fluid sample (2) leakage. Various regions of lyophilized aptamer-MBs or antibody-MBs and aptamer-QDs or antibody-QDs are shown. The reagents, including labeled antibodies or aptamers, etc., are freeze-dried or lyophilized in various adhesive, delimited, or demarcated locations within the microchannels prior to fusing of the two halves (10 and 12). The lower half (10) has a number of channels (18) generally anticipated to be equal in number to the sample ports (14) and are in communication therewith. The channels (18) are sized to be mesofluidic, microfluidic, or nanofluidic. The sample (2), upon introduction into the sample port (14) passes through the port (14) and enters the channel (18). Upon introduction of the sample (2), the sample ports (14) are sealed with a sealant (20), such as tape, plugs, caps, valves, or the like, and the cartridge (4) can be vacuum-packed in an envelope. Vacuum packaging of the cartridges in sealed metallic foil, Mylar, plastic or other airtight envelopes to preserve product freshness and shelf-life over long periods of time. Other embodiments employing capillary action for side or horizontal sample entry are also possible. Also shown is the underlying bar magnet (20) or magnetic field used to pull the MBs (not shown) that are inside the channel (18) along and achieve the rolling sandwich assay. One or more transparent detection windows (16) in the cartridge allow for visual detection or assessment, by a photodetector, camera, or other imaging or photometric device, of the MB bound samples.

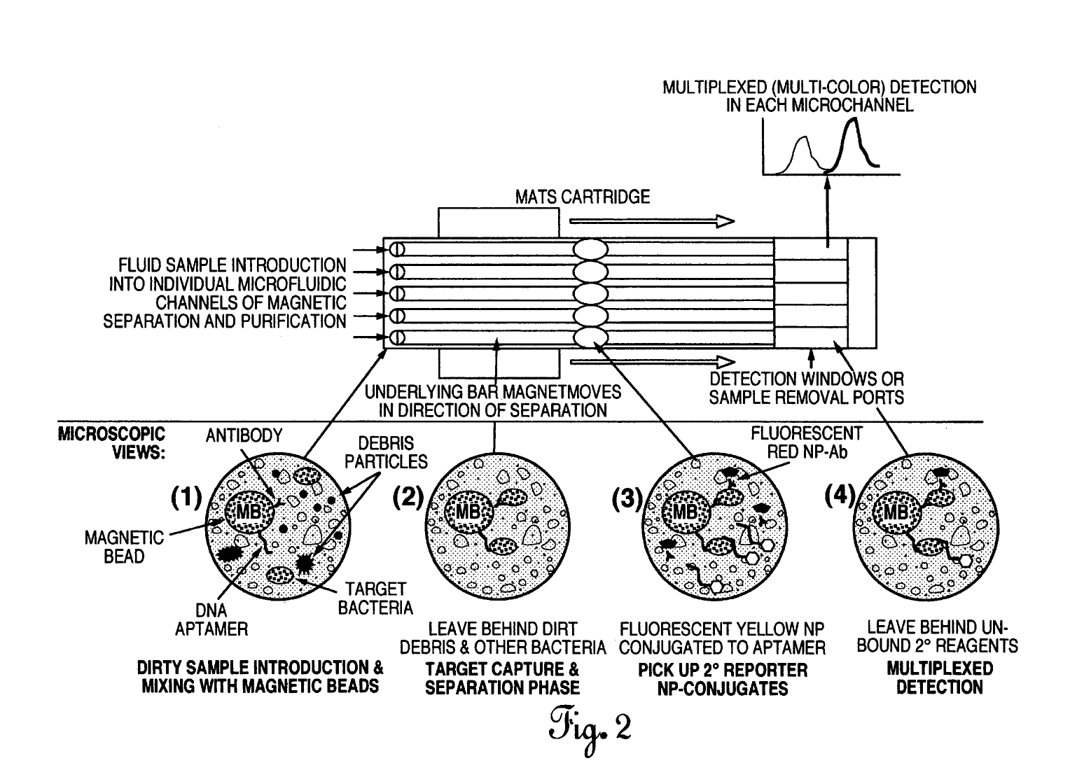

FIG. 2. schematically illustrates the use of the MATS cartridge. A fluid sample (2) is introduced into an individual microfluidic channel (18) through a sample port (14). The target capture and separation phases are then effected. In the channel (18), the sample (2) is introduced to and mixed with a magnetic bead with bound antibody and aptamer. The sample (2) contains debris particles and the target (shown as a bacteria). It is expected that a percentage of the targets will bind with the bound antibody and aptamer. Movement of the MB along the channel (18) in the direction of separation leaves the debris particles behind. The present invention is reminiscent of immunochromatographic test strips employing latex microbeads, except that by using MBs, the operator has control over the flow rate of the particles along their path toward the formation of a sandwich, FRET, or other type of assay on the MB's surface. Control of the MB flow rate is imposed via an external magnet (22), such as a permanent magnet or electromagnet, having a magnetic field that moves the MBs along the channel (18) and halts the MBs wherever critical reagents or sample components must be picked up. The sample and MB can then be introduced to 2° reporter NP-conjugates, such as fluorescent yellow NP or fluorescent red NP-Ab, and bound or conjugated to an aptamer. The MB and sample are then moved to a detection window (16) or sample removal port (16). During this movement, the MB bound sample leaves behind unbound 2° reagents. The detection window (16) or sample removal port (16) allows for multiplexed, or multicolor, detection.

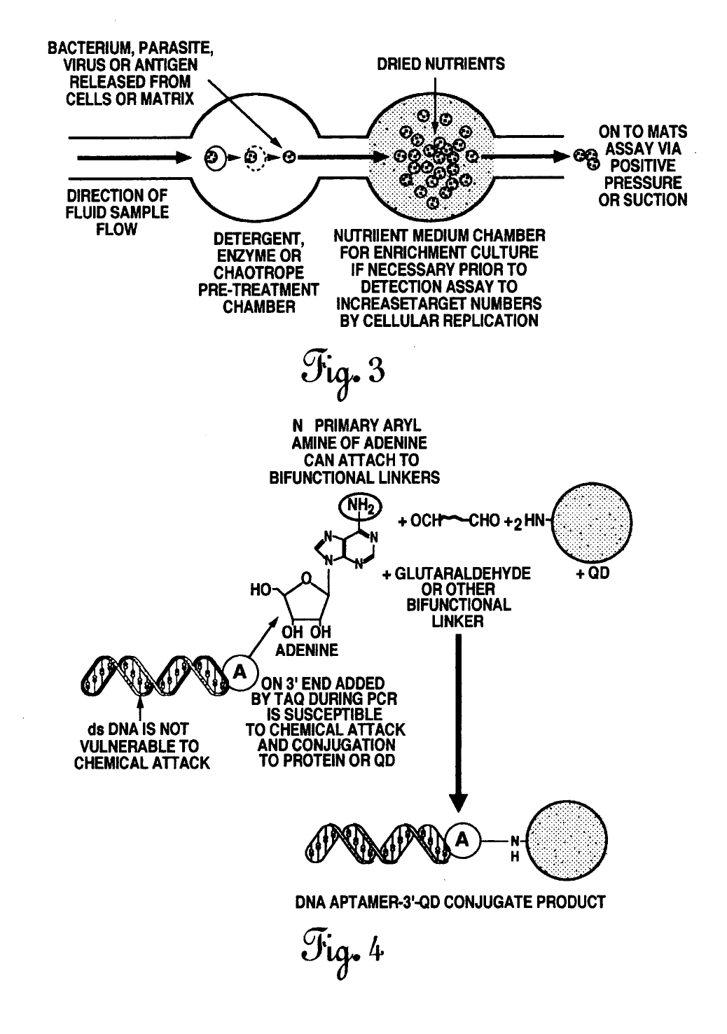

FIG. 3. illustrates an alternative embodiment of the present invention. It illustrates the dissolving and enrichment culture pre-treatment chambers containing dried reagents or nutrients that can be built into the MATS channels prior to the start of the MB-based assays, if necessary. As shown in the figure, cells from the sample can be lysed or stripped of surface antigens by detergents, enzymes, or chaotropes in the first chamber, if desired. This releases the bacterium, parasite, virus, or antigen from the cell or matrix. The sample moves to the second chamber. There, dried culture media or nutrients can be used to increase the numbers of target cells by cell division or proliferation. The two pre-treatment chambers can be used separately, in tandem, or not at all for various applications as needed.

FIG. 4. illustrates N⁶ aryl amine-bifunctional linker attachment chemistry for conjugation of DNA aptamers or other DNA probes after PCR to surface functionalized QDs. Shown on the left is a ds-DNA PCR product having a single unpaired 3′-adenine overhang conferred by Taq polymerase during PCR. This unpaired 3′-adenine is the only base which is vulnerable to attack by glutaraldehyde and other bifunctional linkers, because the rest of the DNA molecule is double-stranded. The adenine is most susceptible to glutaraldehyde or other bifunctional linkers at its primary N⁶ aryl amine position. The glutaraldehyde attacks N⁶ of adenine in a spontaneous reaction that leads to a covalent link between the dsDNA and glutaraldehyde which later attaches covalently to an amine-coated QD as shown. The dsDNA aptamer-QD conjugate can then be made into a functional single-stranded (“ss”) DNA aptamer-QD conjugate by means of heat or urea and purified from byproducts by size-exclusion chromatography. Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A magnetically-assisted test strip apparatus comprising:
a cartridge;
a channel running within said cartridge;
a sample port in communication with said channel, wherein said sample port allows introduction of a fluid sample into said channel; and
said channel containing an aptamer.

2. The apparatus of claim 1, wherein:
said channel contains a magnetic bead; and
said magnetic bead is bound to said aptamer.

3. The apparatus of claim 2, further comprising a magnetic field in proximity to said cartridge so as to influence movement of said magnetic bead along said channel.

4. The apparatus of claim 1, further comprising a detection window in said cartridge providing a view of said channel.

5. The apparatus of claim 2, further comprising a detection window in said cartridge providing a view of said channel.

6. The apparatus of claim 1, wherein said channel is sized to be mesofluidic, microfluidic, or nanofluidic.

7. The apparatus of claim 6, wherein:
said channel contains a magnetic bead; and
said magnetic bead is bound to said aptamer.

8. The apparatus of claim 7, further comprising a magnetic field in proximity to said cartridge so as to influence movement of said magnetic bead along said channel.

9. The apparatus of claim 6, further comprising a detection window in said cartridge providing a view of said channel for visual detection or assessment by a photodetector, camera, or other imaging or photometric device.

10. The apparatus of claim 7, further comprising a detection window in said cartridge providing a view of said channel.

11. The apparatus of claim 1, wherein said cartridge is made from one of: plastic, silica, or metal.

12. The apparatus of claim 3, wherein said magnetic field is generated by a magnet.

13. The apparatus of claim 3, wherein said magnetic field is generated by an electromagnetic field.

14. The apparatus of claim 1, wherein said channel is generally linear.

15. The apparatus of claim 2, wherein said cartridge further comprises a plurality of said channels, wherein a plurality of samples may be processed simultaneously.

16. The apparatus of claim 2, wherein said cartridge further comprises:
a top half; and
a bottom half, wherein said top half and said bottom half are joined via heat, adhesive, mechanical fastener, or other means.

17. The apparatus of claim 2, wherein said cartridge further comprises an enrichment culturing area in said channel containing culture media such as nutrient broth, tryptic soy broth, brain heart infusion broth, other common, or specialized microbiological culture media.

18. The apparatus of claim 2, wherein said magnetically-assisted test strip apparatus further comprises a solubilizing area having ionic or nonionic detergents, enzymes, or chaotropic agents.

19. The method of effecting a sandwich assay in a magnetically-assisted test strip apparatus, comprising:
introducing a target analyte into a channel in said magnetically-assisted test strip apparatus, said magnetically-assisted test strip apparatus comprising a cartridge, said channel running within said cartridge, a sample port in communication with said channel, wherein said sample port allows introduction of a fluid sample containing said target analyte into said channel, said channel containing a magnetic bead, a receptor bound to said magnetic bead, said receptor being a antibody, aptamer, or oligonucleotide, and a detection window;
effecting a first reaction wherein said target analyte binds to said receptor bound to said magnetic bead;
passing magnetic field along length of channel in order to influence said magnetic bead to move along channel to a location viewable through said detection window;
effecting a second reaction wherein said target analyte binds with a reagent, said reagent being chosen from the group: quantum dot, fluorophore-filled nanoparticle, fluorescein-filled nanoparticle, ruthenium trisbipyridine-filled nanoparticle, fluorescent protein, FRET, molecular beacon, colloidal gold, enzyme, labeled antibody, or aptamer;
analysis by means of visual assessment, fluorometer, fluorescence intensity, spectrofluorometry features, fluorescence lifetime analysis, spectrophotometry, spectrofluorometer, time-resolved fluorometer, photodiode, photodiode array, charge-couple device camera, complementary metal oxide semiconductor, photomultiplier tube, or other photodetection device that interfaces with said detection window.

20. The method of claim 19, wherein said magnetically-assisted test strip apparatus further comprises a plurality of channels allowing simultaneous sandwich assays to be performed.

21. The method of claim 19, further comprising lyophilizing said reagent within said channel prior to effecting said other steps of claim 19.

22. The method of claim 19, further comprising vacuum packaging said cartridge in sealed metallic foil, Mylar, plastic or other airtight subsequent to effecting said other steps of claim 19.

23. The method of claim 19, wherein said magnetically-assisted test strip apparatus further comprises an enrichment culturing area in said channel containing culture media such as nutrient broth, tryptic soy broth, brain heart infusion broth, other common, or specialized microbiological culture media.

24. The method of claim 19, wherein said magnetically-assisted test strip apparatus further comprises a solubilizing area having ionic or nonionic detergents, enzymes, or chaotropic agents.
